SEQUENCE LISTING (1 sequence)

SEQ ID NO: 1 - RNA, 22 nt, Homo sapiens
  source 1..22: mol_type = mRNA
tggagtgtgacaatggtgtttg

(19) United States
(12) Patent Application Publication
(10) Pub. No.: US 2025/0304963 A1
(43) Pub. Date: Oct. 2, 2025

(54) REGULATION OF ARTIFICIAL MIRNAS BY ENDOGENOUS TISSUE-SPECIFIC MIRNAS AND METHODS OF USING THE SAME

(71) Applicant: Mirimus, Inc., Brooklyn, NY (US)

(72) Inventor: Giuseppe Militello, New York, NY (US)

(21) Appl. No.: 19/180,809

(22) Filed: Apr. 16, 2025

Related U.S. Application Data
(63) Continuation of application No. PCT/US2023/078541, filed on Nov. 2, 2023.
(60) Provisional application No. 63/382,190, filed on Nov. 3, 2022.

(51) Int. Cl. C12N 15/113

(52) U.S. Cl. CPC C12N 15/113; C12N 2310/113; C12N 2310/531

(57) ABSTRACT

This disclosure relates to introducing specified endogenous amiRNA responsive to endogenous primary-miRNAs. This invention allows the degradation of amiRNAs in specific tissues, while allowing amiRNA to silence gene expression in other tissues. No studies or publications have been found describing a similar approach. Any available amiRNA could be modified and any endogenous miRNA could be used as regulator. The invention is highly modifiable and tunable. The experimental validation pipeline is optimized to enable the discovery of the most functional design. This is done through modifying the loop of a given amiRNA to make it complementary to potentially any endogenous miRNA.

### CROSS-REFERENCE TO RELATED APPLICATIONS

The present application claims priority to PCT application serial no. PCT/US2023/078541 filed on Nov. 2, 2023, which claims priority to U.S. provisional application Ser. No. 63/382,190, filed on Nov. 3, 2022, each herein incorporated by reference in its entirety.

### SEQUENCE LISTING

The present application contains a Sequence Listing which is submitted electronically in XML format and is incorporated by reference in its entirety. Said XML format was created on Apr. 16, 2025, is named Mirimus 106.xml and 1,940 bytes in size.

### BACKGROUND

The present disclosure generally relates to RNAi therapeutics and controlling gene expression through the microRNA (miRNA) biogenesis pathway using artificial microRNA (amiRNA). RNAi therapeutics are potent agents that specifically target a given gene to induce its silencing. One challenging aspect in the employment of this class of therapeutics is to limit the silencing in one or few tissues in the human body. Indeed, silencing in unwanted tissues may result in collateral and adverse effects.

miRNAs are small non-coding RNA molecules, about 21-25 nucleotides (nt) in length, that post-transcriptionally repress gene expression. They are transcribed from miRNA genes in the genome by RNA Polymerase II (RNA Pol II), which yields primordial miRNA (Pri-miRNA). Pri-miRNA will be cleaved by the Drosha-DGCR8 complex, yielding precursor miRNA (Pre-miRNA). Pre-miRNA is then exported to the cytoplasm, where it will be will be further cleaved by a RNase type III enzyme, Dicer, and then unwound by Argonaute 2 protein to yield one mature strand of miRNA.

The mature miRNA can complentarily bind to target mRNA's at the 3′ UTR. By binding to target mRNA's, miRNA can signal to the RNA-induced silencing complex (RISC) to either silence the protein coding region of the mRNA by inhibiting translation if the miRNA imperfectly complements the mRNA, or to degrade the mRNA if the miRNA perfectly complements it. R. Bartoszewski and A. F. Sikorski, “Editorial focus: Understanding off-target effects as the key to successful RNAi therapy,” Cell. Mol. Biol. Lett., vol. 24, no. 1, pp. 1-23, 2019, doi: 10.1186/s11658-019-0196-3.

The present disclosure addresses the issue of silencing in unwanted tissues by utilizing highly expressed endogenous miRNAs in certain cell lines.

### SUMMARY OF THE INVENTION

Provided herein are systems and methods of specific gene silencing. The methods and systems call for the identification of a highly expressed endogenous miRNA in a cell-type, the selection of a base amiRNA sequence which can be modified, maintaining the GUG motif in the amiRNA loop while modifying the amiRNA loop to be complementary to the highly expressed endogenous miRNA, and introducing the modified amiRNA into the target organism. Introduction of the modified amiRNA may silence a gene product post-transcriptionally in a specific cell line. In an embodiment, the loop may be modified to include only six nucleotides that are complementary to the identified miRNA seed sequence and still retain the ability to silence a gene product post-transcriptionally in a specific cell line. In another embodiment, the miR122 responsive element is inserted in the base region of the amiRNA. The method described may be applied to eukaryotic cells, wherein the eukaryotic cell may be human or mammalian. The method may be applied to certain diseases. The method may include a template for amiRNA.

The methods, systems, and apparatuses are set forth in part in the description which follows, and in part will be obvious from the description, or can be learned by practice of the methods, apparatuses, and systems. The advantages of the methods, apparatuses, and systems will be realized and attained by means of the elements and combinations particularly pointed out in the appended claims. It is to be understood that both the foregoing general description and the following detailed description are exemplary and explanatory only and are not restrictive of the methods, apparatuses, and systems, as claimed.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53 (c) EPC and Rule 28 (b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

### BRIEF DESCRIPTION OF THE DRAWINGS

In the accompanying figures, like elements are identified by like reference numerals among the several preferred embodiments of the present invention.

FIG. 1 is an illustration of a hepatocyte that highly expresses miRNA122 and the effect miRNA122 has on introduced amiRNA, which is to degrade the amiRNA.

FIG. 2 is an illustration of a target cell that does not express or scarcely expresses miRNA122 and the effect the absence of miRNA122 has on introduced amiRNA, which is that amiRNA is allowed to aggregate and silence gene expression.

FIG. 3A shows the loop of miRE (an example of amiRNA), specifically the GUG motif, which is important for amiRNA function and should not be modified.

FIG. 3B shows a modification of the loop of miRE to make it complementary to all nucleotides of miR122.

FIG. 3C shows a modification of the loop of miRE to make it complementary to the seed sequence (nucleotides 1 to 6) of miR122.

FIG. 4 shows the miRNA biosynthesis pathway.

FIG. 5 shows a reporter cell that is responsive to the amiRNA. Such cell line will stably express a fluorescent protein (e.g. dTOMATO Red Fluorescent Protein) with a responsive sequence to the amiRNA. The cell line must have low levels of the endogenous miRNA that is supposed to target the amiRNA.

FIG. 6 shows the unmodified amiRNA, as well as the amiRNAs with the modified loop which will be delivered to the reporter cell line. A decrease in fluorescent protein will be observed in all cases but the negative control “no-amiRNA”. If such decrease is not observed in one modification, that should be excluded as the functionality of the amiRNA is abrogated in that case.

FIG. 7 shows the expression of the regulatory endogenous miRNA when forced through transfection of mimic-miRNA or similar techniques. The rescue of fluorescent protein expression is expected as the amiRNA will be degraded. Such rescue is not expected in reporter cells expressing the unmodified amiRNA.

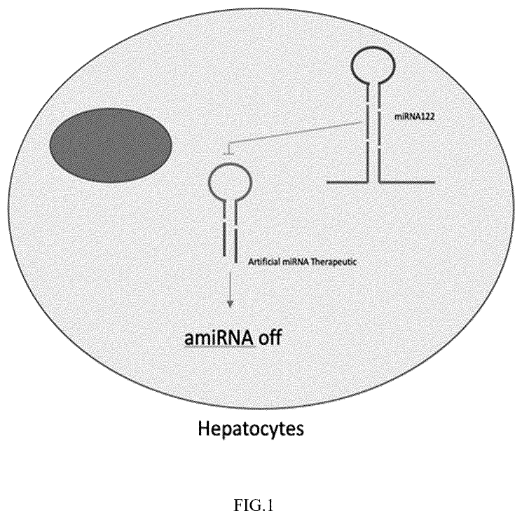

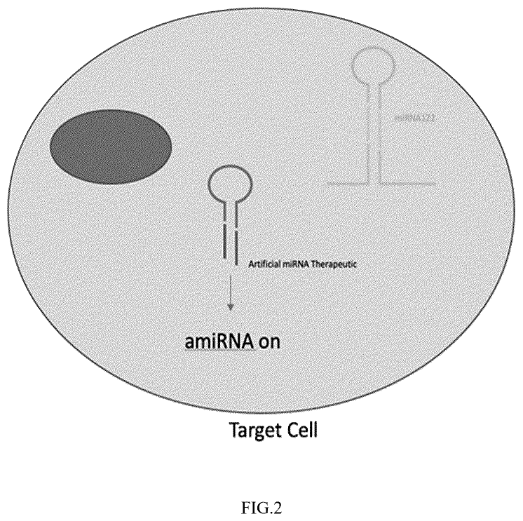

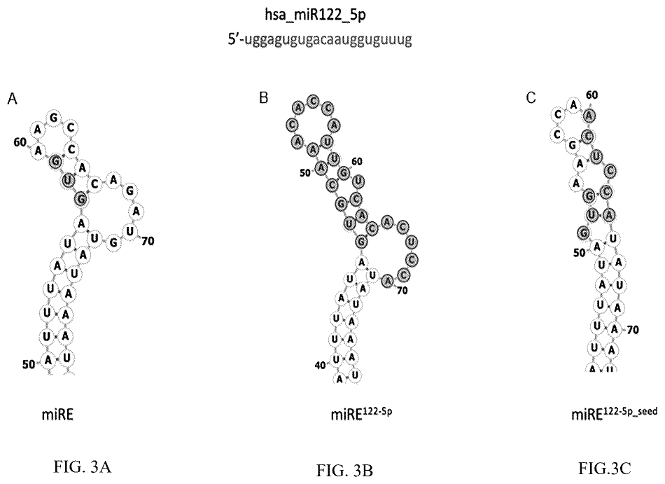

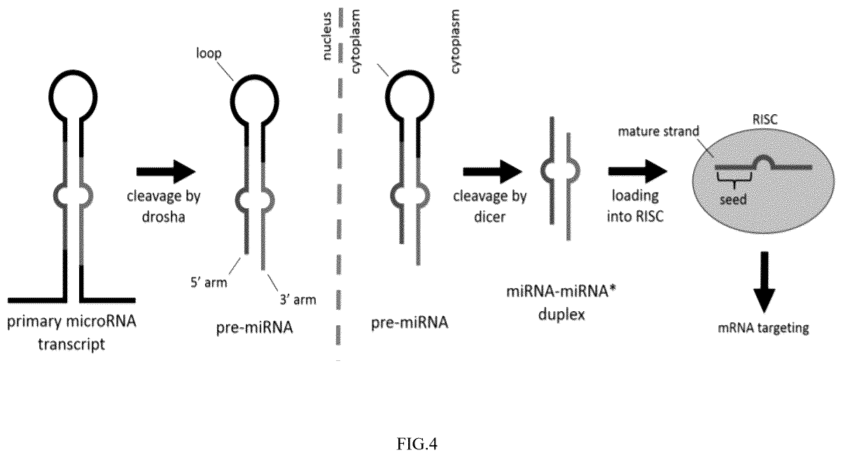

### DETAILED DESCRIPTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### Definitions

The term “complementary” refers to the ability of a nucleic acid to form traditional Watson-Crick or other unconventional types of hydrogen bonds with another nucleic acid sequence. “Imperfectly complements” refers to nucleic acids in amiRNA that mismatch with miRNA nucleic acid sequences yet retain enough complementary sections to bind the strands together.

As used herein, “expression” or “express” refers to the process of transcription from a DNA template into a polynucleotide (e.g., into RNA, mRNA, and miRNA or other RNA transcripts) and/or the subsequent translation of transcribed mRNA into a peptide, polypeptide, or protein. If the polynucleotide is derived from genomic DNA, expression may include splicing of mRNA in eukaryotic cells.

By “post-transcriptionally repress gene expression,” “inhibiting translation,” “inhibition,” or “silencing” are used interchangeably to mean the absence or observable reduction in the level of protein and/or mRNA products from the target gene.

The terms “polypeptide,” “peptide,” and “protein” are used interchangeably herein to refer to a polymer of amino acids of any length. The polymer may be linear or branched, it may comprise modified amino acids, and it may be interrupted by non-amino acids. These terms also encompass amino acid polymers that have been modified. For example, disulfide bond formation, glycosylation, lipidation, acetylation, phosphorylation or any other treatment, such as binding to a labeling component. As used herein, the term “amino acid” includes natural and/or unnatural or synthetic amino acids, including glycine and the right or left stereoisomers, as well as amino acid analogs and peptidomimetics.

The term “seed sequence” refers to an essential sequence for the binding of the miRNA to the mRNA. The seed sequence or seed region is a conserved heptametrical sequence which is mostly situated at positions 2-7 from the miRNA 5′-end.

### DESCRIPTION OF EMBODIMENTS

Provided herein are systems, methods and compositions for specified amiRNA to be introduced to endogenous primary-miRNAs. A given artificial miRNA (amiRNA) can be silenced in specific cell types and tissues by inserting the responsive element of an endogenous miRNA highly expressed in that tissue. In one embodiment, miRNA-122 (miR-122) was chosen as the endogenous miRNA model.

In humans, miR-122 is a miRNA that is highly enriched in the liver but absent from other tissues. miR-122 represents 72% of the miRNA population in cloned sequences. A RNase protection analysis indicates that miR-122 is present at approximately 66,000 copies per cell in adult liver, making it one of the most highly expressed miRNAs in any tissue. The sequence of miR-122 is: 5-UGGAGUGUGACAAUGGUGUUUG-3.

When introduced, the amiRNA in the liver is targeted and degraded by miR-122 which is present in many copies. Oppositely, miR-122 is scarcely present in other cell lines, enabling the accumulation of the amiRNA that can therefore be active and silence the target transcript.

This present disclosure allows the degradation of amiRNAs in specific tissues. No studies or publications have been found describing a similar approach. Any available amiRNA derived from established miRE could be modified and any endogenous miRNA could be used as regulator. The invention is highly modifiable and tunable. The experimental validation pipeline is optimized to enable the discovery of the most functional design.

amiRNAs, similarly to endogenous primary-miRNAs, are formed by base, stem, and loop. In order to harness the RNAi machinery to target a given amiRNA, single-stranded RNA regions should be targeted as they are more likely to be bound by RISC complex harboring the endogenous miRNA complementary to the amiRNAs. Given the fact that the RISC complex is mostly located in the cytoplasm, reason dictates that the loop should be targeted.

The loop of a given amiRNA can be modified to make it complementary to potentially any endogenous miRNA. In order to achieve the optimal silencing, the method comprises: maintaining the GUG motif, modifying the loop to include all complementary nucleotides to the endogenous miRNA of selection; and modifying the loop to include only six nucleotides that are complementary to the seed sequence of the endogenous miRNA of interest.

For maintaining the GUG motif in the loop, this motif is important for the amiRNA function, processing, and downstream efficiency, and thus, should not be modified.

For modifying the loop to include all the complementary nucleotides to the endogenous miRNA of selection, the complementary nucleotides must at least 90% complementary to the endogenous miRNA of selection. In one embodiment, the complementary nucleotides are 91%, 92,%, 93%, 94%, 95%, 96%, 97%, 98%, or 99% complementary to the endogenous miRNA of selection.

For modifying the loop to include only six nucleotides that are complementary to the seed sequence of the endogenous miRNA of interest, this less invasive modification allows a minimal modification of the loop yet allowing silencing as the seed sequence is the most important region for miRNA-target interaction. The pairing of the seed sequence only may not induce degradation of the pre-amiRNA; however, it would cause the RISC complex to bind the pre-amiRNA with subsequent hindering of DICER binding and processing of the pre-amiRNA (steric effect).

The listed modifications will be validated in pre-clinical studies to find out which one of the two yields the best result in terms of inhibition of the amiRNA in the unwanted tissues while still getting a strong amiRNA-dependent silencing in the targeted tissues.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Experimental Validation

Following the design, the following experimental workflow can be employed for validation.

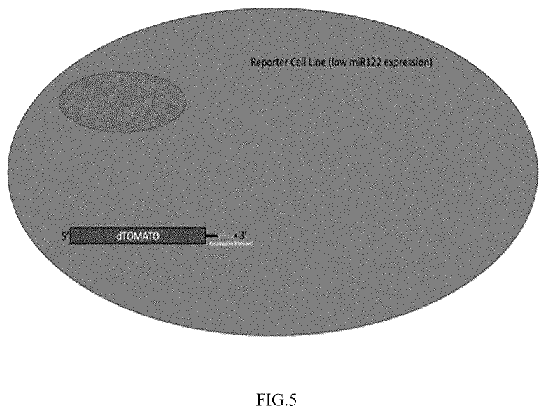

Generate a reporter cell line responsive to the amiRNA. Such cell line will stably express a fluorescent protein (e.g. dTOMATO Red Fluorescent Protein) with a responsive sequence to the amiRNA. Importantly, the cell line must have low levels of the endogenous miRNA that is supposed to target the amiRNA, as shown in FIG. 5.

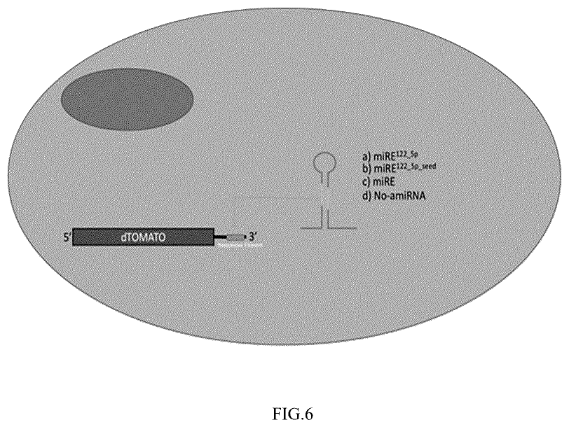

The unmodified amiRNA, as well as the amiRNAs with the modified loop, are delivered to the reporter cell line. A decreasing in fluorescent protein are observed in all cases but the negative control “no-amiRNA”. Importantly, if such decreasing is not observed in one modification, that should be excluded as the functionality of the amiRNA is abrogated in that case, as shown in FIG. 6.

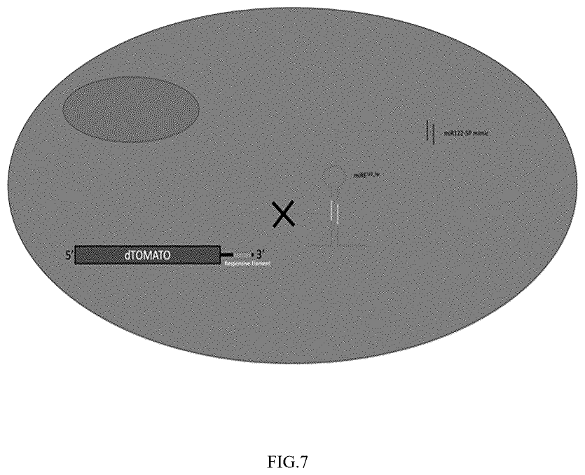

The expression of the regulatory endogenous miRNA is forced through transfection of mimic-miRNA or similar techniques. The rescue of fluorescent protein expression is expected as the amiRNA are degraded. Such rescue is not expected in reporter cells expressing the unmodified amiRNA, as shown in FIG. 7.

### Example 2: AmiRNA Regulation from miRNA Screening

The axis miR122-Liver was used as example to prevent the activation of a given amiRNA in the Liver. The same outcome could be obtained in other tissues if miRNAs that are highly expressed in that tissues are known. The identification of tissue specific miRNAs can be achieved in two different ways:

1) Literature survey to find publications describing the discovery of tissue specific miRNAs; examples: (Deng et al. 2019; Guo et al. 2014; Londina et al. 2015; McCall et al. 2017)

2) Experimental screening in which the investigator isolates total RNA (making sure to retain the small RNA fraction) from: tissue in which the amiRNA should be silenced or tissues in which the amiRNA should be active.

Following RNA isolation, small RNA-seq should be employed to quantify the expression of all endogenous miRNAs; differential expression analysis would then allow to identify miRNAs that are more abundant in the tissue in which the amiRNA must be silenced.

### REFERENCES

Deng, Qipan et al. 2019. “Tissue-Specific MicroRNA Expression Alters Cancer Susceptibility Conferred by a TP53 Noncoding Variant.” Nature communications 10 (1): 5061. http://www.ncbi.nlm.nih.gov/pubmed/31699989.
Guo, Zhiyun et al. 2014. “Genome-Wide Survey of Tissue-Specific MicroRNA and Transcription Factor Regulatory Networks in 12 Tissues.” Scientific Reports 4:1-9.
Londina, Eric et al. 2015. “Analysis of 13 Cell Types Reveals Evidence for the Expression of Numerous Novel Primate- And Tissue-Specific MicroRNAs.” Proceedings of the National Academy of Sciences of the United States of America 112 (10): E1106-15.
McCall, Matthew N. et al. 2017. “Toward the Human Cellular MicroRNAome.” Genome Research 27 (10): 1769-81.

All publications and patent applications mentioned in this specification are herein incorporated by reference to the same extent as if each individual publication or patent application was specifically and individually indicated to be incorporated by reference.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A method of specific gene silencing, comprising the steps of:
a) identifying a highly expressed endogenous miRNA in a cell-type;
b) selecting a base amiRNA sequence to modify, maintaining the GUG motif in the loop;
c) modifying an amiRNA loop to be complementary to the highly expressed endogenous miRNA; and
d) introducing the modified amiRNA into the target organism.

2. The method of claim 1, wherein a gene product is post-transcriptionally silenced in a specific cell line.

3. The method of claim 1, wherein the loop is modified to include only six nucleotides that are complementary to the identified miRNA seed sequence.

4. The method of claim 1, wherein (application to certain diseases).

5. The method of claim 1, wherein (template for amiRNA is chosen from a list of . . . ).

6. The method of claim 1, wherein the amiRNA sequence is optimized for gene silencing in eukaryotic cells.

7. The method of claim 6, wherein the eukaryotic cell is human or mammalian.

8. The method of claim 1, wherein the base is modified to include only six nucleotides that are complementary to the identified miRNA seed sequence.

9. A modified amiRNA produced by claim 1.

10. A modified amiRNA produced by claim 3.
